(19) United States
(12) Patent Application Publication
(10) Pub. No.: US 2004/0096069 A1
(43) Pub. Date: May 20, 2004

(54) Electronic stethoscope

(76) Inventor: Jen-Chien Chien, Yi-Lan Hsien (TW)

Correspondence Address: TROP PRUNER & HU, PC, 8554 KATY FREEWAY, SUITE 100, HOUSTON, TX, 77024, US

(21) Appl. No.: 10/294,960

(22) Filed: Nov. 14, 2002

(51) Int. Cl. A61B 7/04; A61B 7/02; A61B 5/02; A61B 7/00

(52) U.S. Cl. 381/67; 600/528; 600/586; 181/131

(57) ABSTRACT

In an electronic stethoscope, a filter unit receives and filters an audio signal attributed to a human body part and received by a receiver so as to output a set of target signals associated with the audio signal. The target signals have frequencies within specific frequency bands and are converted into a set of digital signals. An operating unit is operable so as to generate an input mode signal corresponding to a selected operating mode for selecting one of the digital signals. A processor receives the set of digital signals and the input mode signal, and processes and outputs one of the digital signals that is selected according to the input mode signal. An audio playback unit receives and reproduces output of the processor.

### BACKGROUND OF THE INVENTION

1. Field of the Invention

The invention relates to a stethoscope, more particularly to an electronic stethoscope.

2. Description of the Related Art

FIG. 1 illustrates a conventional stethoscope 1 that includes a Y-shaped tube 11, a sound receiver 12, and two earpieces 13. The flexible Y-shaped tube 11 has a main tube part 111 connected to the sound receiver 12, and two branch parts 112 connected to the earpieces via two ear tubes 14. The sound receiver 12 is coupled electrically to the earpieces 13 via conducting wires passing through the Y-shaped tube 11 and the ear tubes 14. The sound receiver 12 picks up sounds generated by a human body part, such as heart, lungs and intestines, so as to generate an audio signal corresponding to the sounds to the earpieces 13. The earpieces 13 receive the audio signal and generate an audio output corresponding to the audio signal.

When medical personnel utilize the conventional stethoscope 1 to hear activity sounds of a human body part of a patient, noises in the environment cannot be avoided. Furthermore, if a patent inappropriately speaks or breathes during diagnosing through the conventional stethoscope 1, the audio output containing noises from the environment would result in inaccurate diagnosis.

Moreover, since the sound receiver 12 of the conventional stethoscope 1 usually picks up sounds having frequencies within a specific frequency band, additional sound receivers capable of receiving sounds that have frequencies within various frequency bands are needed, thereby resulting in relatively high costs and inconvenience during use.

### SUMMARY OF THE INVENTION

Therefore, the object of the present invention is to provide an electronic stethoscope that can easily and accurately process an external audio signal according to a selected operating mode at a relatively low cost.

According to the present invention, an electronic stethoscope comprises:

a receiver adapted for receiving an external audio signal attributed to a human body part;

a filter unit coupled electrically to the receiver for receiving and filtering the audio signal received by the receiver so as to output a set of target signals associated with the audio signal, each of which has a frequency within a specific frequency band;

an analog-to-digital converter coupled electrically to the filter unit for converting the set of the target signals into a set of digital signals;

an operating unit operable so as to generate an input mode signal corresponding to a selected operating mode for selecting one of the digital signals;

a processor coupled electrically to the analog-to-digital converter and the operating unit for receiving the set of digital signals and the input mode signal therefrom, the processor processing and outputting one of the digital signals that is selected according to the input mode signal; and

an audio playback unit coupled electrically to the processor for receiving and reproducing output of the processor.

### BRIEF DESCRIPTION OF THE DRAWINGS

Other features and advantages of the present invention will become apparent in the following detailed description of the preferred embodiment with reference to the accompanying drawings, of which:

FIG. 1 is a perspective view showing a conventional stethoscope;

FIG. 2 is a schematic electrical block diagram showing the preferred embodiment of an electronic stethoscope according to the present invention; and

FIG. 3 is a perspective view showing the preferred embodiment.

### DETAILED DESCRIPTION OF THE PREFERRED EMBODIMENT

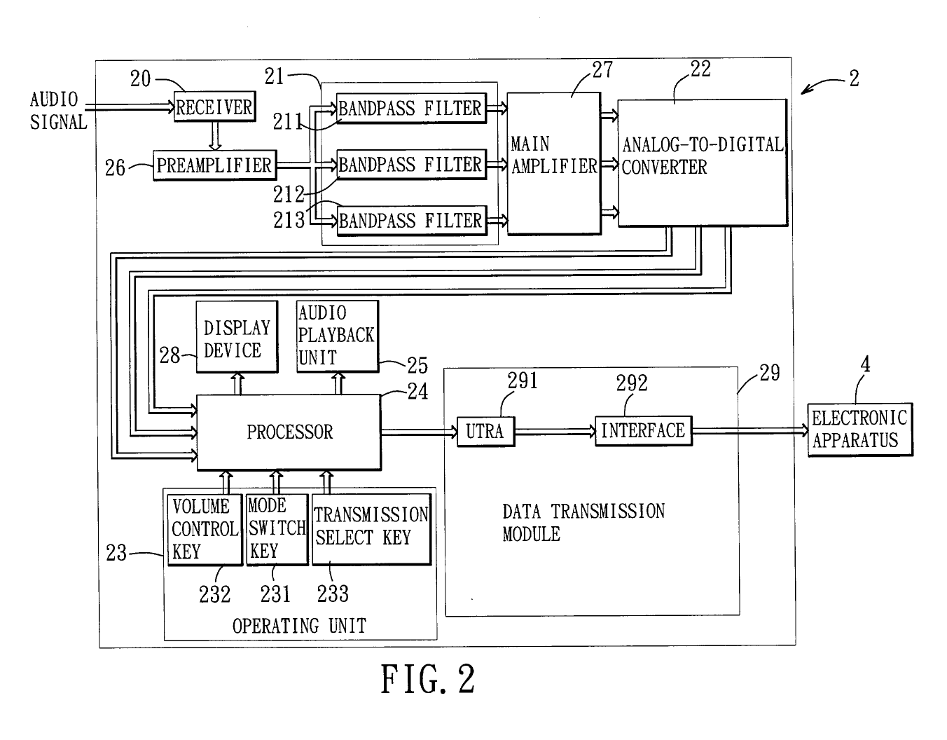

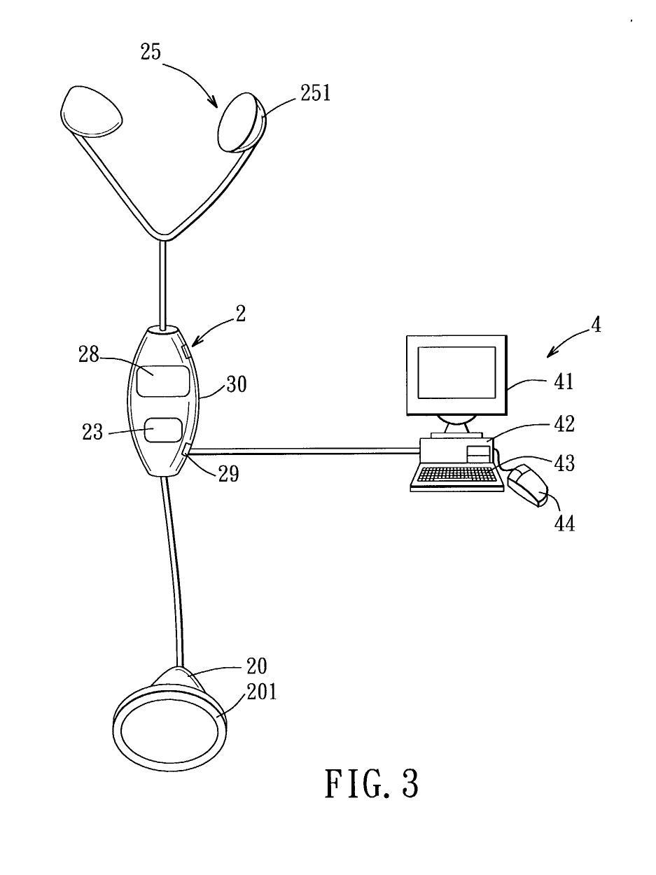

Referring to FIGS. 2 and 3, the preferred embodiment of an electronic stethoscope 2 according to the present invention is shown to include a receiver 20, a filter unit 21, an analog-to-digital converter 22, an operating unit 23, a processor 24, and an audio playback unit 25.

The receiver 20 is adapted for receiving an external audio signal attributed to a human body part, such as the heart, the lungs or the intestines. In this embodiment, the receiver 20 is a known electrical condenser microphone provided with a disk-type pick-up head 201 thereon for sound collection, as shown in FIG. 3. Generally, sounds generated by biological activity of the heart have frequencies within a frequency band of about 20˜230 Hz, sounds generated by biological activity of the lungs have frequencies within a frequency band of about 100˜800 Hz, and sounds generated by biological activity of the intestines have frequencies within a frequency band of about 20˜500 Hz. As such, the audio signal received by the receiver 20 has a frequency within a frequency band of 20˜800 Hz.

A filter unit 21 is coupled electrically to the receiver 20 for receiving and filtering the audio signal received by the receiver 20 so as to output a set of target signals associated with the audio signal. Each of the target signals has a frequency within a specific frequency band. In this embodiment, the filter unit 21 includes a set of bandpass filters 211, 212, 213, each of which includes a low-pass filter and a high-pass filter (not shown). The bandpass filter 211, which is suited for filtering the audio signal attributed to the human heart, has a frequency band of 20˜230 Hz, i.e., the low-pass filter of the bandpass filter 211 has a cut-off frequency of 230 Hz for filtering noises having frequencies higher than 230 Hz, and the high-pass filter of the bandpass filter 211 has a cut-off frequency of 20 Hz for filtering noises having frequencies lower than 230 Hz such that aliasing errors can accordingly be prevented. Similarly, the bandpass filter 212, which is suited for filtering the audio signal attributed to the human lungs, has a frequency band of 100˜800 Hz. The bandpass filter 213, which is suited for filtering the audio signal attributed to the human intestines, has a frequency band of 20˜500 Hz.

The preferred embodiment of the electronic stethoscope 2 of this invention further includes a preamplifier 26 coupled electrically to the receiver 20 and the filter unit 21 for amplifying the audio signal received by the receiver 20 such that quantization errors due to weak audio signals, such as the audio signal attributed to the human lungs, can be avoided.

The analog-to-digital converter 22 is coupled electrically to the filter unit 21 for converting the set of target signals into a set of digital signals. Furthermore, the electronic stethoscope 2 further includes a main amplifier 27 coupled electrically to the filter unit 21 and the analog-to-digital converter 22 for amplifying the target signals from the filter unit 21 to distinguish apparently the target signals from noise.

In this embodiment, the operating unit 23, which is mounted on a housing 30, includes a mode switch key 231, a volume control key 232 and a transmission select key 233. The mode switch key 231 is operable so as to generate an input mode signal corresponding to a selected operating mode, which is one of a first operating mode for diagnosis of the heart, a second operating mode for diagnosis of the lungs, and a third operating mode for diagnosis of the intestines, for selecting one of the digital signals.

The processor 24 is coupled electrically to analog-to-digital converter 22 and the operating unit 23 for receiving the set of the digital signals and the input mode signal therefrom. The processor 24 processes and outputs one of the digital signals that is selected according to the input mode signal. That is, when the operating unit 23 generates the input mode signal corresponding to the first operating mode, the processor 24 processes and outputs one of the digital signals that corresponds to the target signal from the bandpass filter 211. When the operating unit 23 generates the input mode signal corresponding to the second operating mode, the processor 24 processes and outputs one of the digital signals that corresponds to the target signal from the bandpass filter 212. When the operating unit 23 generates the input mode signal corresponding to the third operating mode, the processor 24 processes and outputs one of the digital signals that corresponds to the target signal from the bandpass filter 213.

The audio playback unit 25 is coupled electrically to the processor 24 for receiving and reproducing output of the processor 24. In this embodiment, as shown in FIG. 3, the audio playback unit 25 includes two earpieces 251. The volume control key 232 is operable so as to generate a control signal such that the processor 24 is able to control volume of the output reproduced at the audio playback unit 25.

Moreover, a display device 28, such as a liquid crystal display, is mounted on the housing 30 and is coupled electrically to the processor 24 for displaying information in the selected operating mode. For example, when the electronic stethoscope 2 is operated in the first operating mode, characters such as “heart” are shown on the display device 28; when the electronic stethoscope 2 is operated in the second operating mode, characters such as “lungs” are shown on the display device 28; or when the electronic stethoscope 2 is operated in the third operating mode, characters such as “intestines” are shown on the display device 28. Thus, information shown on the display device 28 indicates the selected operating mode so as to facilitate use.

Furthermore, in order to obtain accurate diagnosis, an external electronic apparatus is utilized for analyzing the output of the processor 24. Therefore, the electronic stethoscope 2 further includes a data transmission module 29 coupled electrically to the processor 24 and adapted to be coupled electrically to an external electronic apparatus 4. The data transmission module 29 is adapted to transmit the output of the processor 24 to the electronic apparatus 4 for analysis. In this embodiment, the data transmission module 29 includes a universal asynchronous receiver transmitter (UART) 291, coupled electrically to the processor 24, and an interface 292, such as RS232, universal serial bus, IEEE1394, infrared rays, Bluetooth or the like, coupled electrically to the UART 291 and adapted to be coupled electrically to the electronic apparatus 4. The UART 291 converts the output of the processor 24 into data that has a format suitable for transmission through the interface 292. The transmission select key 233 of the operating unit 23 is operable so as to generate a control signal to the processor 24 such that the processor 24 is able to transmit the output thereof to the electronic apparatus 4. As shown in FIG. 3, the electronic apparatus 4 is a personal computer that includes a main module 41, a display 42, and an input device having a keyboard 43 and a mouse 44.

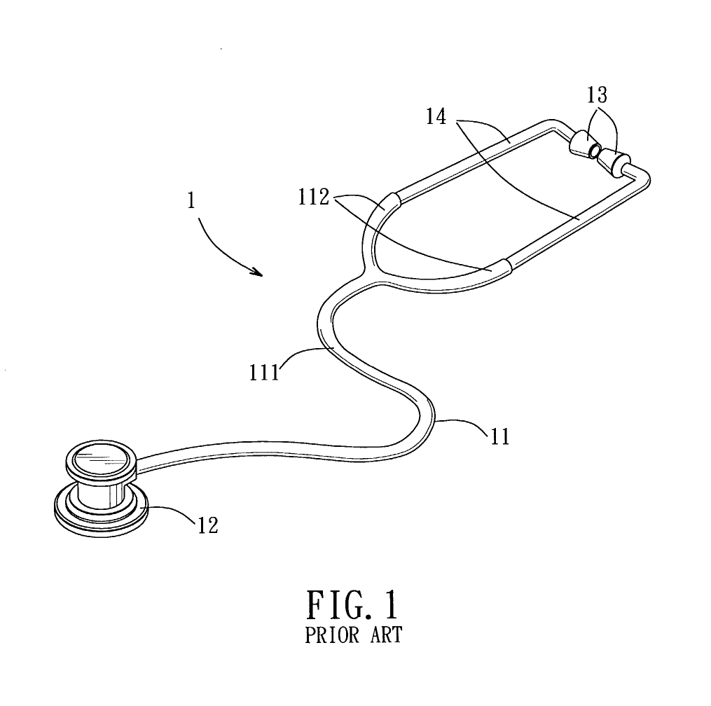

In sum, in this embodiment, when the electronic stethoscope 2 of this invention is activated to diagnose a human body part, the receiver 20 receives an external audio signal attributed to the human body part. The audio signal is amplified by the preamplifier 26 and is then filtered by the filter unit 21 such that three target signals are outputted. Subsequently, the target signals are amplified by the main amplifier 27 and are converted into three digital signals by the analog-to-digital converter 22. The processor 24 further processes the digital signals, e.g., filters noises from the digital signals, and outputs one of the digital signals according to an input mode signal from the operating unit 23, that corresponds to a selected one of the first, second and third operating modes, to the audio playback unit 25 such that activity sounds of the human body part can be heard by a user wearing the earpieces 251. When the electronic stethoscope 2 is switched to another one of the first, second and third operating modes through operation of the mode switch key 231, the processor 24 enables the display device 28 to show information corresponding to the selected operating mode and further enables the audio playback unit 25 to reproduce the output of processor 24. When compared to the aforesaid conventional stethoscope 1 of FIG. 1, due to the presence of the filter unit 21 and the operating unit 23, the electronic stethoscope 2 of this invention permits diagnosis of different body parts at a relatively low cost.

It is noted that the filter unit 21 is not limited to include only three bandpass filters 211, 212, 213. For example, an additional bandpass filter with a specific frequency band for a specific human body part, such as joints, may be included in the filter unit of the stethoscope of this invention.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

I claim:

1. An electronic stethoscope comprising:
a receiver adapted for receiving an external audio signal attributed to a human body part;
a filter unit coupled electrically to said receiver for receiving and filtering the audio signal received by said receiver so as to output a set of target signals associated with the audio signal, each of which has a frequency within a specific frequency band;
an analog-to-digital converter coupled electrically to said filter unit for converting the set of the target signals into a set of digital signals;
an operating unit operable so as to generate an input mode signal corresponding to a selected operating mode for selecting one of the digital signals;
a processor coupled electrically to said analog-to-digital converter and said operating unit for receiving the set of digital signals and the input mode signal therefrom, said processor processing and outputting one of the digital signals that is selected according to the input mode signal; and
an audio playback unit coupled electrically to said processor for receiving and reproducing output of said processor.

2. The electronic stethoscope as claimed in claim 1, wherein said filter unit includes a set of bandpass filters, each of which includes a low-pass filter and a high-pass filter.

3. The electronic stethoscope as claimed in claim 1, wherein said filter unit includes a bandpass filter having a frequency band of 20˜230 Hz.

4. The electronic stethoscope as claimed in claim 1, wherein said filter unit includes a band-pass filter having a frequency band of 100˜800 Hz.

5. The electronic stethoscope as claimed in claim 1, wherein said filter unit includes a band-pass filter having a frequency band of 20˜500 Hz.

6. The electronic stethoscope as claimed in claim 1, further comprising a preamplifier coupled electrically to said receiver and said filter unit.

7. The electronic stethoscope as claimed in claim 1, further comprising a main amplifier coupled electrically to said filter unit and said analog-to-digital converter.

8. The electronic stethoscope as claimed in claim 1, further comprising a display device coupled electrically to said processor for displaying information in the selected operating mode.

9. The electronic stethoscope as claimed in claim 1, wherein said receiver is an electrical condenser microphone.

10. The electronic stethoscope as claimed in claim 1, further comprising a data transmission module coupled electrically to said processor and adapted to be coupled electrically to an external electronic apparatus, said data transmission module being adapted to transmit the output of said processor to the electronic apparatus for analysis.
